(19) United States
(12) Patent Application Publication
(10) Pub. No.: US 2010/0131024 A1
(43) Pub. Date: May 27, 2010

(54) ELECTROTHERAPY ORTHOPEDIC DEVICE

(76) Inventors: Peter Lathrop, San Diego, CA (US); Joel Smith, San Diego, CA (US)

Correspondence Address: CATALYST LAW GROUP, APC, 9710 SCRANTON ROAD, SUITE S-170, SAN DIEGO, CA, 92121, US

(21) Appl. No.: 12/444,362

(22) PCT Filed: Oct. 5, 2007
(86) PCT No.: PCT/US07/80597

§ 371 Date: Dec. 28, 2009

Related U.S. Application Data
(60) Provisional application No. 60/828,508, filed on Oct. 6, 2006.

(51) Int. Cl. A61N 1/00

(52) U.S. Cl. 607/3

(57) ABSTRACT

An electrotherapy orthopedic device including an electronic capsule and an orthopedic device, together providing a means to supply electrotherapy to damaged bones and tissue or promoting bone ingrowth to the device. A pre-programmed microprocessor delivers an optimal electrotherapy treatment regime, wherein a sequence of pulses form a plurality of waveforms available for specific clinical needs. A waveform, its pulse frequency, pulse width, duration and intensity (in sum, “Waveform Parameters”) are pre-programmed into the microprocessor based on the optimal treatment regimen for any particular disorder.

### PRIORITY CLAIM

This patent application contains subject matter claiming benefit of the priority date of U.S. Provisional Patent Application Ser. No. 60/828,508 filed on Oct. 6, 2006 and entitled ELECTROTHERAPY ORTHOPEDIC DEVICE, accordingly, the entire contents of this provisional patent application is hereby expressly incorporated by reference.

### BACKGROUND OF THE INVENTION

Clinical electrotherapy devices are generally known and are used to implement many different types of human medical therapy protocols. Electrotherapy devices may be used to stimulate nerve cells and other forms of tissues in the human body to achieve a large number of therapeutic ends. In addition, electrical impulses cause muscles to contract and may be used for various forms of exercise, rehabillitation and pain management. These electrotherapy devices are generally referred to as Transcutaneous Electrical Nerve Stimulator (TENS) devices.

TENS and other microcurrent and/or millicurrent electrotherapy stimulation devices have been used successfully for the symptomatic relief and management of chronic intractable pain and other disorders for many years. While TENS is primarily intended for pain relief via a nerve signal blocking mechanism, it has also been used to promote healing via reduction of inflammation and the appropriate release of biochemicals. TENS is also used to treat or maintain disorders including, but not limited to, carpal tunnel syndrome and viral infections such as herpes, as well as treating open wounds.

One effective TENS-based treatment is interferential therapy, is generally described in T. W. Wing, Interferential Therapy: How it Works and What's New, The Digests of Chiropractic Economics, May/June 1992. Effective interferential therapy depends on proper electrode placement and a synchronized firing of all the electrodes to create optimal electrode interplay, it is the current state of the art, that interferential therapy is generally reserved for inpatient treatments, wherein a highly skilled therapist arranges four or eight electrodes and manually adjusts a TENS unit to deliver interferential therapy.

A wide variety of TENS devices have been developed and are generally described in U.S. Pat. No. 6,023,642 by Shealy et al., U.S. Pat. No. 5,620,470 by Gliner et al., U.S. Pat. No. 5,607,454 by Cameron et al., and U.S. Pat. No. 5,601,612 by Gliner et al. These TENS devices are large and cumbersome, complex to operate, expensive and require lead wires running to each electrode, making them difficult for use at home, at work or at play.

Previous attempts have been made to design improved electrotherapy devices, certain features of which are generally described in U.S. Pat. No. 6,083,250 to Lathrop; U.S. Pat. No. 6,445,955 to Michelson et al.; U.S. Pat. No. 5,620,470 to Gliner et al.; U.S. Pat. No. 5,607,454 to Cameron et al.; U.S. Pat. No. 5,607,461 to Lathrop; U.S. Pat. No. 5,601,612 to Gliner et al.; U.S. Pat. No. 5,593,427 to Gliner et al.; U.S. Pat. No. 5,584,863 to Rauch et al.; U.S. Pat. No. 5,578,060 to Pohl et al.; U.S. Pat. No. 5,573,552 to Hansjurgens; U.S. Pat. No. 5,549,656 to Reiss; U.S. Pat. No. 5,514,165 to Malaugh et al.; U.S. Pat. No. 5,476,481 to Schondorf; U.S. Pat. No. 5,387,231 to Sporer; U.S. Pat. No. 5,397,338 to Grey et al.; U.S. Pat. No. 5,374,283 to Flick; U.S. Pat. No. 5,354,320 to Schaldach et al.; U.S. Pat. No. 5,304,207 to Stromer; U.S. Pat. No. 5,183,041 to Toriu et al.; U.S. Pat. No. 5,133,352 to Lathrop et al.; U.S. Pat. No. 4,989,605 to Rossen; U.S. Pat. No. 4,759,368 to Spanton et al.; U.S. Pat. No. 4,699,143 to Dufresne et al.; U.S. Pat. No. 4,398,545 to Wilson; and U.S. Pat. App. Pub. No. 2005/0049654 A1.

Specifically, U.S. Pat. App. Pub. No. 2005/0049654 A1 to Lathrop, a joint inventor herein, is directed to an ultralight preprogrammed microprocessor based electrotherapy device without the need of adjustment by the user. While many devices heretofore have provided appreciable advancements to the art, no devices or methods have attempted to apply this technology to orthopedic applications. More specifically, prior art devices have applied micro-current stimulation to topical areas of the body, however, no device has applied TENS therapy directly to deep tissue, joints, bones, tendons and other portions of the human musculoskeletal system.

In light of the above, it is an object of the present invention to provide an electrotherapy device and method that specifically targets rehabilitation of bones, joints and skeletal muscle. It is yet another object of the present invention to provide an electrotherapy orthopedic device capable of delivering various types of electrotherapy.

### BRIEF SUMMARY OF THE INVENTION

Generally, the present invention provides an electrotherapy orthopedic device including an electronic capsule, comprising a switchable power supply, a pre-programmed microprocessor, at least two electrodes, and at least two wires; and an orthopedic device, together providing a means to supply electrotherapy to damaged bones and tissue. The pre-programmed microprocessor delivers an optimal electrotherapy treatment regime, wherein a sequence of pulses form a plurality of waveforms available for specific clinical needs. A waveform, its pulse frequency, pulse width, duration and intensity (in sum, “Waveform Parameters”) are pre-programmed into the microprocessor based on the optimal treatment regimen for any particular disorder.

In use, the electrotherapy orthopedic device of the current invention, will provide optimized electrotherapy treatment regimens to the damaged bone and tissue. In one embodiment, the electrotherapy orthopedic device provides standard electrotherapy. In another embodiment, the electrotherapy orthopedic device provides interferential therapy. And in a further embodiment the electrotherapy orthopedic device provides three-dimensional quadripolar, interferential, micro-current stimulation.

### BRIEF DESCRIPTION OF THE FIGURES

FIG. 1 is a cross sectional illustration of the electrotherapy orthopedic device incorporated into a bone wherein the orthopedic device is an intermedullary nail.

FIG. 2 is a cross sectional illustration of the electrotherapy orthopedic device incorporated into a bone, with exploded cutaways of the bone and fixation device revealing a schematical illustration of the electronic circuit.

FIG. 3 is a cross sectional schematic illustration of the electrotherapy orthopedic device wherein the orthopedic device is a plate.

FIG. 4 is an isometric illustration of one embodiment of the current invention having four electrodes and further illustrating current direction in interferential therapy.

### DETAILED DESCRIPTION OF THE INVENTION

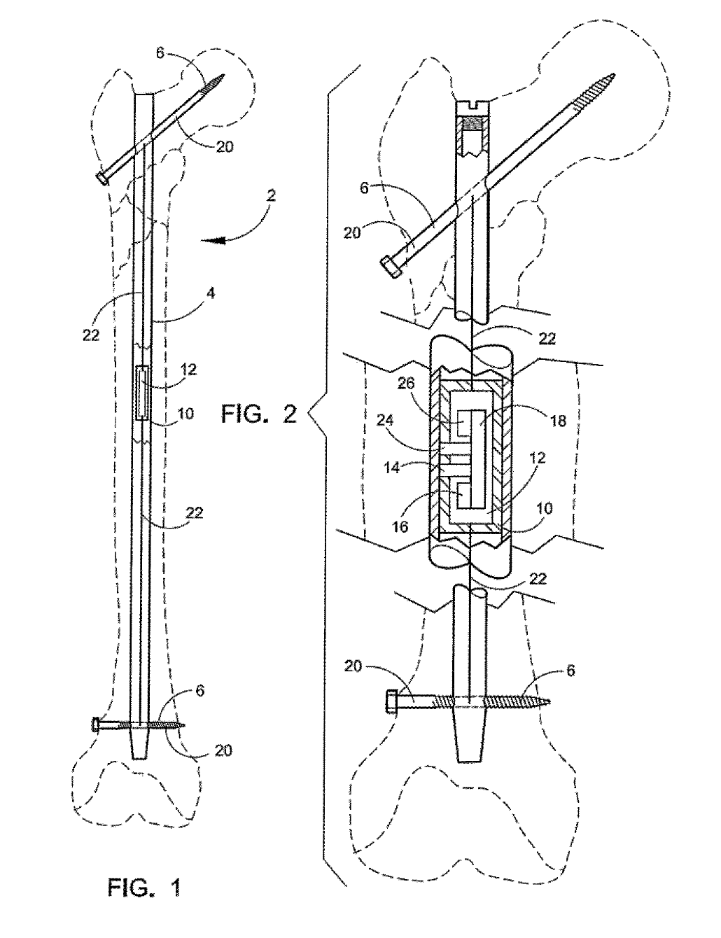

Turning now to the drawings, in which like numerals indicate like elements throughout the several views. FIG. 1 shows that the current invention is directed towards an electrotherapy orthopedic device (herein “EOD”) 2 that can be implanted into a bone, thereby allowing the user to comfortably receive electrotherapy treatments which increases the healing and recovery process of a bone and/or surrounding tissue.

The current invention is capable of delivering an optimal electrotherapy treatment regimen, wherein optimal Waveform Parameters (defined as pulse frequency, pulse width, pulse amplitude, modulation, duration and intensity) are pre-programmed into the invention microprocessor 18 and are optimally delivered to treat specific injuries. As used in this disclosure of the current invention, the term “ . . . microprocessor being pre-programmed with Waveform Parameters for delivering optimal electrotherapy treatment regimens” refers to the programming of the microprocessor 18 of the EOD 2 to deliver an optimal electrotherapy. In the art of electrotherapy, the Waveform Parameters can be adjusted during therapy. Depending on the required treatment, the Waveform Parameters are adjusted at specific times, thereby delivering a changed current from time point A to time point B to time point C . . . etc. Such Waveform Parameter settings and time points for changing are known in the art. The settings of the Waveform Parameters must be precisely accurate in order to deliver optimal electrotherapy. Additionally, the time to change these Waveform Parameters is similarly critical to delivery of an optimal electrotherapy. Thus, these changes in Waveform Parameters and the critical time points can be programmed into the microprocessor 18 in order to deliver the precise and optimal therapy as is known in the art, and in this context the term “ . . . microprocessor being pre-programmed with Waveform Parameters for delivering optimal electrotherapy treatment regimens” is used.

Delivered current can be of any amperage however milliamperage (millicurrent) and microamperage (microcurrent) are more commonly employed. By way of the pre-programmed microprocessor, the inherent errors and time lapse associated with user or therapist operation is eliminated. The Waveform Parameters of the current invention are determined, delivered and changed by command of the microprocessor 18 in a cycle that has been pre-programmed into said microprocessor 18 and which is based on the known optimal Waveform Parameters treatments in the electrotherapy arts. Pre-programming the treatment Waveform Parameter cycles into the microprocessor allows the microprocessor to deliver accurate and time sensitive electrotherapy without interference by a user or therapist who would otherwise have to adjust the unit.

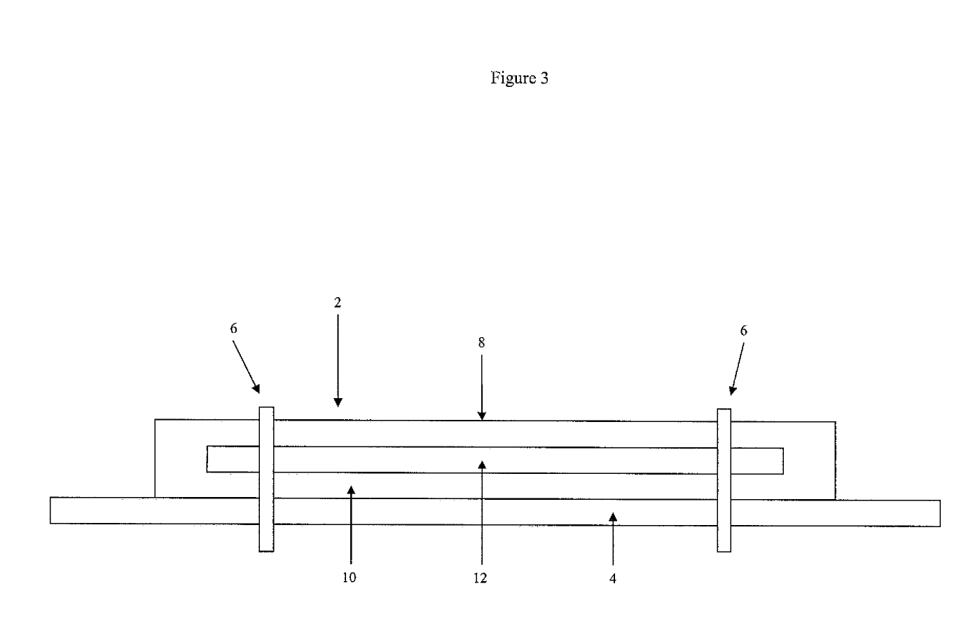

EOD 2 of the present invention comprises an orthopedic fixation device (herein “OFD”) 4 and an electronics capsule 8. The EOD 2 can take the shape of any OFD 4 known in the art including, but not limited to, intramedullary nails, spinal rods, plates, hinges, or any shape suitable for embodying the current invention. FIG. 3 displays an EOD 2 comprising on OFD 4 and an electronics capsule 8, wherein the OFD 4 is a plate.

The current invention can utilize all forms of an OFD 4 towards bones including, but not limited to: an intramedullary nail into a femur, tibia, or humerus; a plate onto an epiphyseal fracture, metaphyseal fracture, or diaphyseal fracture; and a spinal rod onto a posterior fusion mass stimulation or an anterior intervertebral body fusion in the spine. In addition, the current invention can utilize all forms of an OFD 4 towards total joint replacements including, but not limited to, the hip (i.e. the femoral stem and acetabular component), the knee (i.e. femoral component and tibial component) the ankle (i.e. the tibial component and talar component), the elbow (i.e. the humeral component and ulnar component), and the shoulder (i.e. the glenoid component and the humeral component).

In FIG. 2, the electronics capsule 8 comprising a protective environment 10 and an electronics circuit 12 is illustrated. Methods for encapsulating the electronics circuit 12 with the protective environment 10 are well known in the art. In such an embodiment, the protective environment 10 inhibits electric current from traveling to the OFD 4 which prevents shorting of the electronics circuit 12. The composition of the protective environment 10 is an electric non-conductive material including, but not limited to, polyethylene, silicones, silastic materials, latex, etc.

In the preferred embodiment, electronics capsule 8 is slid through an open end of an OFD 4 into the hollow shaft of the OFD 4. Electronics capsule 8 is then held into place by securing an end cap onto the open end of the OFD 4. In an alternative embodiment, wherein OFD 4 is a spinal rod or a plate, electronics capsule 8 would rest on top of the OFD 4. In such an embodiment, electronics capsule 8 can be secured to OFD 4 via bone fixation hardware (herein “BFH”) 6 wherein the BFH is a screw that runs through the electronics capsule 8 and is secured to the rod/plate, the bone, or both the rod/plate and bone. The BFH 6 can include, but is not limited to a nail, screw, bolt, or any other fastening device appropriate for this embodiment.

Electronics circuit 12, shown in FIG. 2, is optimally encapsulated within the protective environment 10. Preferably, the electronics circuit 12 comprises: switchable power supply 14; a power supply 16; a pre-programmed microprocessor 18; at least two electrodes 20; and at least two wires 22. Switchable power supply 14 is preferably a mechanical single pole dual throw (herein “SPDT”) switch that controls power delivered to the electronics circuit 12. Alternatively, switchable power supply 14 is an open electronics circuit 12 when in the off position. In this alternative embodiment, an exposed portion of the electronics circuit 12 has an electrical gap. When the EOD 2 comprising an electronics capsule 8 and an OFD 4 is surgically incorporated into the patient's body the circuit is closed by way of the electrical conductivity of said patient's body. By way of example only, the electrical gap could be an exposed and broken wire. Those of skill in the art will readily design gap circuits for use in the current invention.

Power supply 16 is electrically connected to the electronics circuit 12. In a preferred embodiment, power supply 16 is prescribed to deliver power to provide electrotherapy for a specified amount of time. The power supply 16 can take the form of all different durations of time depending on the type of treatment required. The power supply 16 can include, but is not limited to, a battery or any other power supply appropriate for this embodiment.

Pre-programmed microprocessor 18 is electrically connected to the electronics circuit 12. In a preferred embodiment, pre-programmed microprocessor 18 is pre-programmed to deliver a series of currents having the optimal Waveform Parameters to treat a number of disorders responsive to electrotherapy. Pre-programmed microprocessor 18 is pre-programmed to determine, deliver and change these Waveform Parameters in a cycle and range that is based on the optimal Waveform Parameters for optimally treating numerous disorders, thereby eliminating the need to adjust these same Waveform Parameters as prescribed in a given treatment protocol after the device is surgically implanted. Electrotherapy treatment protocols, including the Waveform Parameters and cycle times, are well known by those of ordinary skill in the art. By removing user facilitated adjustments of the Waveform Parameters from the treatment regimen, the current invention is also necessarily removing inherent user errors, such as delays and inaccuracies in shifting from one set of Waveform Parameters to another, as is generally required in electrotherapies. By way of the pre-programmed parameter cycle, the device precisely sets the Waveform Parameters to the prescribed setting, and does so at the precise optimal time, thus delivering the most efficient and optimal treatment and thereby reducing time and costs of treatment.

The power generated when switchable power supply 14 is activated is controlled by the pre-programmed microprocessor 18 to deliver optimal Waveform Parameters to the electrodes 20. Each electrode is electrically connected by way of an insulated wire 22 from the electronics circuit 12 to the electrodes 20. Electrodes and electrically conductive materials forming electrodes are well known to those of ordinary skill in the art. In the preferred embodiment in which the OFD 4 is an intramedullary nail, the electrodes 20 is a BFH 6 that secures the intramedullary nail to the bone. The insulated wire 22 runs from the electronics circuit 12, through the hollow cavity of the intramedullary nail, and connects to the BFH 6 of the intramedullary nail 4 rendering the BFH 6 an electrode 20.

In the preferred embodiment, insulated wire 22 ends in an eyelet which is positioned flush with the hole of the intermedullary nail 4. As the electrodes 20 is inserted through the intermedullary nail 4, the electrodes 20 passes through the eyelet of the insulated wire 22 thereby connecting the wire 22 to the electrodes 20. In such an embodiment, the electrodes 20 is insulated with a non-conductive material thereby preventing the passage of current between the electrodes 20 and the intermedullary nail 4. In a preferred embodiment, the insulation comprises two grommets in which the eyelet of insulated wire 22 is positioned between the two grommets and both the grommet holes and insulated wire 22 eyelet holes are positioned flush with the holes of the intermedullary nail 4 thereby allowing the electrodes 20 to pass through the first grommet, the eyelet, and the second grommet. The grommets prevent the passage of current from the electrodes 20 or insulated wire 22 to the intermedullary nail 4 which in turn prevents the shorting of the circuit.

In yet another embodiment, the outer-surface of the insulated wire 22 eyelet can be insulated with non-conductive material. The two openings allowing the electrodes 20 to pass through the intermedullary nail 4 are also insulated with non-conductive material. The non-conductive material covering both the insulated wire 22 eyelet and the openings of the intermedullary nail 4 prevent the passage of current from the electrodes 20 or insulated wire 22 to the intermedullary nail 4 which in turn prevents shorting of the circuit.

In yet another embodiment, the electrodes 20 is an end cap in which the insulated wire 22 runs from the electronics circuit 12, through the hollow cavity of the OFD 4 and connects to the end cap thereby rendering the end cap an electrode. In this embodiment, a washer made of non-conductive material is installed between the OFD 4 and the end cap thereby inhibiting the passage of current between the end cap and the OFD 4. Additionally, the end cap can be a wire or any other device appropriate for this embodiment.

Additionally, where the OFD 4 is a spinal rod or a plate, the electrodes 20 is a BFH 6 that secures the electronics capsule 8 to the plate and/or bone in which BFH 6 passes through protective environment 10, electronics circuit 12, OFD 4, and into the damaged bone rendering the BFH 6 an electrode. In such an embodiment, the BFH 6 is insulated with a non-conductive material at the section of the BFH 6 where the BFH 6 contacts the OFD 4. The non-conductive material prevents the passage of current between OFD 4 and BFH 6. Specifically, the non-conductive material can be a grommet positioned in the opening of the OFD 4 preventing the electrodes 20 from contacting the OFD 4 which in turn prevents shorting of the circuit.

While the preferred number of electrodes 20 is two, the number of electrodes 20 is not restricted to two and is determined by the type of electrotherapy required to heal the broken bone or damaged tissue. In addition, the electrodes 20 can be located at any position on the OFD 4. However, the preferred location of the electrodes 20 is that closest to the broken bone of the damaged tissue.

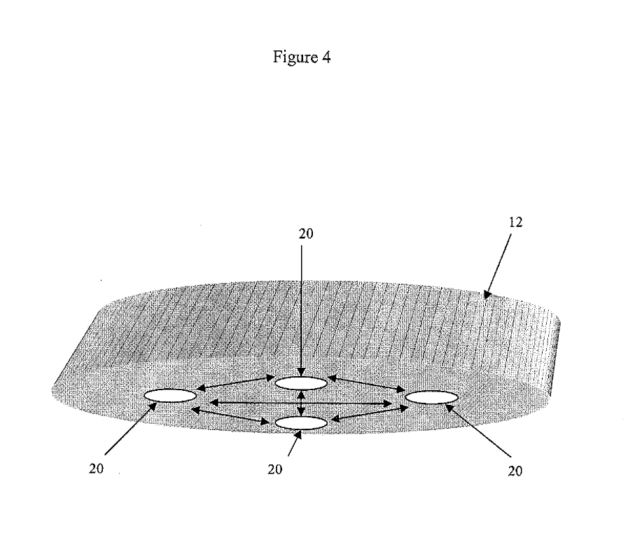

In a preferred embodiment, electronics circuit 12 comprises at least two electrodes 20. In an alternative embodiment shown in FIG. 4, electronics circuit 12 comprises a plurality of electrodes comprising an interferential electronics circuit (herein “IEC”). The IEC comprises at least two independent circuits and at least two independent sets of electrodes. The combination of such electrodes creates at least three different frequencies all of which can provide simultaneous electrotherapy. In the IEC embodiment wherein electronics circuit 12 comprises a plurality of electrodes, each of the electrodes 20 is capable of acting as an anode and a cathode. The electrodes' 20 polarity is driven by the electronics circuit 12, which synchronizes the polarity of the electrodes 20 to alternately deliver and/or receive electrotherapy current. In one example, illustrated in FIG. 4, electronics circuit 12 comprises four electrodes 20. In this example, the pre-programmed microprocessor 18 of the electronics circuit 12 directs each of the electrodes 20 to deliver a bipolar interferential therapy. In the IEC embodiment, the at least two of the four electrodes 20 are synchronized to deliver and receive current (depicted as dual head arrows in the figure) from the diagonal and adjacent electrodes. In order to accomplish an effective interferential therapy, the polarity switch in each of the two sets of two electrodes 20 is precisely timed to deliver to its proper electrode partner.

Further to FIG. 2, the electronics capsule 8 may also comprise an LED display 24, which is visible from the exterior surface of electronics capsule 8. LED display 24 is responsive to the power delivery generated by said SPDT switch, thereby notifying the user of the on/off status of said BUD 2. In addition, the LED display 24 allows the user to ensure that the electronics circuit 12 is receiving power. LED display 24 is electrically connected to electronics circuit 12 on one end, traverses the electronics capsule 8, and is visible on the external surface of the OFD 4, thereby being visible to a user for the determination of the electrical current status of EOD 2. For example, the LED display 24 lights up when switchable power supply 14 is in the “ON” position, indicating to the user that a current is running through said system. When switchable power supply 14 is in the “OFF” position, LED display 24 will not light up. An additional advantage to LED display 24 is that a user can monitor whether the EOD 2 is working properly. Because many optimal electrotherapy treatments will utilize microcurrents, a user will not be able to determine whether the device is functional based on electrical sensation. Thus, LED display 24 will indicate to a user whether an electrical current is being discharged by EOD 2 when switchable power supply 14 is in the “ON” position.

In addition, the current invention may comprise a wireless communication transceiver 26, which is electrically connected to electronics circuit 12. The wireless communication transceiver 26 communicates with a remote controller held by the user. The wireless communication transceiver 26 allows the user to adjust the electrotherapy by adjusting specific parameters of the electrotherapy including, but not limited to, Waveform Parameters.

In this embodiment, the user's remote controller will generate a digital and/or analogue signal, and will relay this signal to the wireless communication transceiver 26, which is also electrically coupled to electronics circuit 12. The user's remote is also electrically coupled to an antenna, and will transmit a digital signal using said antenna. The transmitted digital signal will be received by antenna of the wireless communication transceiver 26. The antenna is electrically coupled to the wireless communication transceiver 26, which in turn is electrically coupled to the electronics circuit member 12. The digital and or analogue message is received by electronics circuit 12, the instructions are interpreted and a proper response is implemented by the electronics circuit member 12. The electronics circuit member 12 of the at least one remote slave unit 6 is further described in detail, below.

Digital and/or analogue messages wirelessly transmitted between the user remote and the wireless communication transceiver 26 include, but are not limited to; messages regarding the treatment regimen and associated Waveform Parameters for a particular treatment regimen; the timing and setting adjustments of the Waveform Parameters, which are made when delivering said optimal treatment regimen; and synchronization of the electrical discharge patters to deliver said optimal treatment regimens. The user's remote controller, via the wireless communication transceiver 26, directs the electronics circuit 12 regarding how and when to perform any particular electrotherapy regimen.

In the preferred embodiment, the EOD 2 comprises an OFD 4 and an electronics circuit 12 in which the electronics circuit 12 is installed into the OFD 4 by a user immediately prior to the installation of the EOD 2 into the patient. In an alternative embodiment, EOD 2 comprises an OFD 4 and an electronics circuit 12 in which the EOD 2 is manufactured in such a manner that the electronics capsule 8 is a non-removable component of the EOD 2. Here the electronics capsule 8 is incorporated into the EOD 2 at the manufacturing stage thereby eliminating the need for the user to incorporate the electronics capsule 8 into the EOD 2 immediately prior to surgical implantation.

In an alternative embodiment, the electronics circuit 12 is implanted into the patient absent the OFD 4. The electronics circuit 12 could be implanted under the skin or into the tissue of a patient thereby avoiding the highly invasive procedure of implantation of an orthopedic device.

The invention will be further illustrated by reference to the following non-limiting examples. In the following examples, EOD 2 is pre-programmed to deliver Waveform Parameters that will optimally treat pain. However, it should be noted that the EOD 2 of the current invention can be implemented and used for treatment of any disorder, wound, ailment or other condition that is treatable using electrotherapy. For example, treatment regimens can include, but are not limited to: carpal tunnel syndrome, arthritis, insomnia, soft tissue repair, muscle strain and inflammation. For each treatment regimen, the pre-programmed microprocessor 18 will have a set of instructions to deliver the optimal treatment according to a treatment protocol well known in the art. Electrotherapy treatment Waveform Parameters are known by those of skill in the art, and are generally disclosed in the literature (For pain, see e.g., Sjolund, B., et al. (1977) Acta Physiol. Scand., 100: 382-384; Kim. J. H. K., et al., (1984) Brain Res., 304: 192-196; Pert, C. B., et al., (1973) Science, 182: 1359-1361; Sjolund, B., et al. (1978) Second World Congress of Pain Abstracts, Vol. 1: 15; von Knorring, L., et al. (1978) Pain, 5: 359; Myer, D. J., et al. (1976) Pain 2: 379-404, Hokfelt, T., et al. (1977) Proc. Natl. Acad. Sci. USA, 74: 3081-3085; Mudge, A. W., et al. (1979) Proc. Natl. Acad. Sci. USA, 76: 525-530; Pert, C. B., et al., (1973) Science. 179: 1011-1014; Sjolund, B., et al. (1976) The Lancet, II: 1085.)

### Example 1

In this example, the EOD 2 of the current invention is used to treat a broken femur and damaged tissue. A patient requiring electrotherapy treatment for a broken femur and damaged tissue will use the EOD 2 whereas the OFD 4 and the electronics capsule 8 are separate components of the EOD 2, the OFD 4 is an intramedullary nail, and the BFH 6 is a screw.

The user will first install the electronics capsule 8 into the hollow shaft of the OFD or intramedullary nail, via the open end of the intramedullary nail. The user will then secure an end cap to the open end of the intramedullary nail thereby holding the electronics capsule 8 into place. The user inserts the intramedullary nail into the broken bone and inserts the BFH 6, or screws, through the intramedullary nail and into the broken femur, thereby securing the intramedullary nail to the broken femur. Upon securing the screws, the screws connect to the insulated wires 22 via the eyelet and become the electrodes 20. Upon securing, the EOD 2 to the patient, the switchable power supply 14 is activated thereby releasing power from the power supply 16 to the electronics circuit 12 and the LED display 24. The electronics circuit 12 will operate to deliver electrotherapy to the treatment area, thereby beginning the healing process of the afflicted area.

Electrotherapy will continue for the duration of the prescribed power supply 16 at which time the electronics circuit 12 will cease to perform electrotherapy treatment.

### Example 2

In this example, the EOD 2 of the current invention is used to treat an epiphyseal fracture. A patient requiring, electrotherapy treatment for epiphyseal fracture will use the EOD 2 whereas the OFD 4 and the electronics capsule 8 are components integrated together during manufacturing of the EOD 2, the OFD 4 is a plate (FIG. 3), the BFH 6 is a screw and the electronics circuit 12 is an interferential electronics circuit (IEC). The IEC comprises two independent circuits and two independent sets of at least two electrodes 20 for a total of four electrodes 20. The combination of such electrodes creates at least three different frequencies all of which provide simultaneous electrotherapy.

As the electronics capsule 8 is incorporated into the OFD 4 during the manufacturing process, the user will not need to install the electronics capsule 8 into the OFD 4. The user will apply the OFD 4, or plate onto the broken bone. The user will then insert the BFH 6, or screws, through the electronics capsule 8, protective environment 10, the plate, and into the broken bone. Upon securing the screws, the screws connect to the electronic circuit 12 and become the electrodes 20. Upon securing the EOD 2 to the patient, the switchable power supply 14 is activated thereby releasing power from the power supply 16 to the electronics circuit 12 and the LED display 24. The electronics circuit 12 will operate to deliver electrotherapy to the treatment area, thereby beginning the healing process of the afflicted area. The pre-programmed microprocessor 18 of the electronics circuit 12 will begin to cycle through and thereby deliver electrotherapy to each of the four electrodes 20 the Waveform Parameters optimally suited to treat an epiphyseal fracture. In addition, electronics circuit 12 will operate to deliver interferential micro-current stimulation to the treatment area.

Electrotherapy will continue for the duration of the prescribed power supply 16 at which time the electronics circuit 12 will cease to perform electrotherapy treatment.

What is claimed:

1. An electrotherapy orthopedic device (2) comprising:
an orthopedic fixation device (4); and
an electronics capsule (8);
whereas the orthopedic fixation device provides the infrastructure to conduct electrotherapy and the electronic capsule provides there current required to conduct electrotherapy towards broke bones and damaged tissue.

2. An electrotherapy orthopedic device of claim 1, the electronics capsule comprising:
a protective environment (10); and
an electronics circuit (12);
whereas the protective environment inhibits the passage of electric current from the electronics circuit to the orthopedic fixation device and the electronics circuit provides the current required to conduct electrotherapy towards broke bones and damaged tissue.

3. An electrotherapy orthopedic device of claim 2 wherein the electronics circuit comprises:
a switchable power supply (14);
a power supply (16);
a pre-programmed microprocessor (18);
at least two electrodes (20); and
at least two wires (22).

4. An electrotherapy orthopedic device of claim 3 wherein the electronics circuit further comprises an LED (24).
